(19) United States
(12) Patent Application Publication
(10) Pub. No.: US 2011/0129546 A1
(43) Pub. Date: Jun. 2, 2011

(54) DERMATOLOGICAL PHARMACEUTICAL COMPOSITION FOR THE TREATMENT OF SKIN INFLAMMATION DISEASES, SUCH AS DERMATITIS, ATOPIC DERMATITIS, VITILIGO, ALOPECIA AREATA, ACNE, PSORIASIS, PRURITUS OR COMBINATIONS OF SAME

(76) Inventor: Ignacio Umbert Mill, Barcelona (ES)

(21) Appl. No.: 12/920,832

(22) PCT Filed: Jun. 19, 2009
(86) PCT No.: PCT/ES2009/000339

§ 371 Date: Dec. 9, 2010

(30) Foreign Application Priority Data
Jun. 20, 2008 (ES) P20081861

(51) Int. Cl. A61K 31/405; A61K 31/56; A61K 31/522; A61K 36/82; A61K 36/18; A61K 33/32; A61K 36/886; A61K 31/496; A61K 31/7036; A61K 31/7056; A61K 31/63; A61K 31/7048; A61K 31/4406; A61P 17/00; A61P 17/06; A61P 17/10; A61P 17/04; A61P 29/00; A61P 39/06; A61P 31/00; A61P 31/10

(52) U.S. Cl. 424/639; 514/420; 514/171; 514/263.32; 424/729; 424/769; 424/744; 514/254.07; 514/40; 514/253.08; 514/24; 514/155; 514/29; 514/355

(57) ABSTRACT

The invention relates to a dermatological pharmaceutical composition for the treatment of skin inflammation diseases, such as dermatitis, atopic dermatitis, vitiligo, alopecia areata, acne, psoriasis and pruritus. The invention comprises a base anti-inflammatory agent, such as indometacin; one or more optional active ingredients selected alternatively from among at least a corticoid and an antibiotic; and a combination of topical antioxidants used to potentiate the anti-inflammatory effect, selected from among green tea, lipoic acid, curcumin, ascorbyl palmitate, Coenzyme Q10, resveratrol, Pycnogenol™, L-camosine, taurine, vitamin E, vitamin C, papaya extract, isoflavones, manganese, lycopene and quercetin. At least one of the topical antioxidants is a peroxisome proliferator-activated receptor-gamma (PPAR-γ) activator. The invention also includes at least one antioxidant substance with an antiproliferative effect on keratonocytes, e.g. manganese, and at least one substance that blocks tumour necrosis factor-alpha (TNF-α) or other cytokines that provoke the acute phase of the inflammatory reaction, also with an antiproliferative effect, e.g. pentoxifylline.

### TECHNICAL FIELD OF INVENTION

The present invention relates to a pharmaceutical composition for treating dermatological inflammatory diseases of the skin, such as for example dermatitis, atopic dermatitis, vitiligo, alopecia areata, acne, psoriasis and pruritus, and combinations of them, which contain at least one anti-inflammatory base, such as indometacin.

### BACKGROUND OF THE INVENTION

In dermatology, today there are frequent multiple inflammatory diseases of neurogenic origin. These diseases can be dermatitis, atopic dermatitis, vitiligo, alopecia areata, acne, psoriasis and pruritus, etc., according to immunological alterations, gene activations, infections, destruction of melanocytes destruction of immunoprivilege of the hair follicle, etc. occur.

There are currently multiple embodiments of drugs for the treatment of each of these diseases, mainly based on the use of an anti-inflammatory, such as indometacin, ibuprofen (although this is very photosensitive), their equivalents, or corticoids, topically.

In dermatology it is not usual the administration of combinations of active ingredients. This is generally because of the difficulty to found by the person skilled in the art the combination of two or more active ingredients, with respect to the chemical stability and interactions that can cause drug products to be present in the same formulation (see FR248454 of L'Oreal).

There is therefore in dermatology the perception that the association of active ingredients is generally not effective against skin diseases.

Therefore, the formulations against inflammatory diseases in dermatology are based on anti-inflammatories, corticoids alone or combined (for example, with doxepin, for example in CN1363276) of powerful action, not without side effects.

The present invention aims to disclose a pharmaceutical compound that overcomes this barrier, and is suitable for a very effective treatment of many diseases, concomitant or not, of inflammatory kind, mainly but not exclusively, arising from new known etiologies, for example allostatic overload by neuroimmunoendocrine stress.

### SUMMARY OF THE INVENTION

To this end, the object of the present invention is a new pharmaceutical composition for treating dermatological inflammatory diseases of the skin, such as for example dermatitis, atopic dermatitis, vitiligo, alopecia areata, acne, psoriasis and pruritus comprising an anti-inflammatory such as indometacin, characterized in that it further comprises
a combination of topical antioxidants to boost the anti-inflammatory effect by the activation of the peroxisome proliferator-activated receptor gamma (PPAR-γ); and
one or more optional active ingredients selected alternately from:
at least one corticoid; and
an antibiotic.

The compositions according to the invention also comprise preferably at least one antioxidant substance with keratinocyte anti-proliferative effect, and a blocking substance of tumor necrosis factor alpha (TNF-α), or other cytokines stimulant of inflammatory reactions, also with anti-proliferative effects.

The preferred antioxidant substance with keratinocytes anti-proliferative effect is manganese, particularly in the case of psoriasis, and the preferred blocking substance of tumor necrosis factor alpha (TNF-α), also with anti-proliferative effect is pentoxifylline. Manganese is part of enzymes such as superoxide dismutase SOD, which show a high antioxidant capacity and protection against free radicals, with anti-proliferative effect.

Manganese and pentoxifylline are included in weight proportions of up to 5%.

According to another feature of the present invention, at least one of the topical antioxidants is an activator of the peroxisome proliferator-activated receptor gamma (PPAR-γ), such as lipoic acid.

In a first variant of the drug, designed particularly for the treatment of dermatitis, atopic dermatitis, vitiligo, alopecia areata, psoriasis and pruritus, said optional active ingredients are corticoids.

In a second variant, corticoids are replaced by an antibiotic for the treatment of acne, as inflammatory process of the skin.

In case of corticoids, clobetasol propionate can be used, especially for the treatment of vitiligo, alopecia, areata, and psoriasis, in a decreasing concentration with decrease of the pathological intensity, preferably between 0.1 and 0% by weight.

For the treatment of dermatitis and atopic dermatitis, corticoids are preferably a combination of hydrocortisone and triamcinolone acetonide, particularly in a decreasing concentration with decrease in the pathological intensity, between 0.5 and 0% by weight,

According to another feature of the present invention, in the first variant doxepin can be added, especially for the treatment of atopic dermatitis, especially between 0 and 5% by weight.

Topical antioxidants are selected from: quercetin, catechins from green tea, lipoic acid, curcumin, ascorbyl palmitate, Coenzyme 010, resveratrol, pycnogenol®, L-carnosine, taurine, vitamin E, vitamin C, papaya extract, isoflavones and lycopene. Manganese is also expected to be use as an antioxidant in the case of psoriasis.

The preferred combinations of topical antioxidants of formulations of the present invention are:
5% ascorbyl palmitate, 5% vitamin E, and 3% lycopene,
1% lycopene, 5% vitamin E, and 3% vitamin C,
1% resveratrol,
3-5% lipoic acid,
Coenzyme Q10 3%, 5% vitamin E, and 5% taurine,
C.s.p. % papaya extract,
0.5% green tea catechins,
1% pycnogenol®, 3% vitamin C, and 5% ascorbyl palmitate,
1% curcumin,
2% L-carnosine, 3% vitamin C, 5% vitamin. E, and 5% ascorbyl palmitate,
5% isoflavones, 0.5% green tea catechins; 5% ascorbyl palmitate, and
1% quercetin
   that the medical practitioner will select depending on the intensity of the inflammatory disease.

The dermatological pharmaceutical composition of the present invention may further comprise
an emollient selected from: glycerin, aloe vera, propylene glycol, and lactic acid
an adjunctive of anti-inflammatory, such as omega-3,
an antifungal such as ketoconazole, and
L-carnitine.

In the case of the first variant, with corticoids, the pharmaceutical composition according to the invention can also include an antibiotic such as gentamicin, ciprofloxacin, clindamycin, or equivalents.

In the case of the second option, free of corticoids, the antibiotic is preferably selected from the group consisting of: ciprofloxacin, clindamycin, sodium sulfacetamide, gentamicin and erythromycin.

In both cases, the drug according to the invention can also contain nicotinamidae.

The new dermatological pharmaceutical compositions of the present invention are completely inventive, because until now, all would suggest a dermatologist skilled in the art that the association of these components would have an adverse or anti-synergistic effect.

The effects of a combination of antioxidants are a stimulation of the activation of PPAR-γ and an increase of the anti-inflammatory efficacy. Thus, generally the resulting formulations of the present invention can reduce the weight proportions of aggressive substances such as steroids, antibiotics and effective doxepin, while each of these ingredients acts on one or a few specific diseases, potentiating surprisingly, possibly by a synergistic effect, the anti-inflammatory effect and allowing the formulation in accordance with the intensity of each disease,

### DESCRIPTION OF PREFERRED EMBODIMENTS

The object of the invention is a dermatological drug product useful for the treatment of inflammatory diseases of the skin, such as for example dermatitis, atopic dermatitis, vitiligo, alopecia areata, acne, psoriasis, pruritus, etc., comprising the following active ingredients,
a base anti-inflammatory, such as indometacin,
one or more antioxidants with anti-proliferative effect of keratinocytes,
a TNF-α anti-proliferative and blocking substance (or other cytokines that produce inflammations)
an active ingredient selected from:
an antibiotic for the treatment of acne, as inflammatory process of the skin, and
one or more corticoids for the rest of diseases, and
a combination of topical antioxidants, enhancers of the activation of PPAR-γ.

Suitable emollients and excipients are also added.

The preferred anti-proliferative antioxidant is manganese, especially useful for psoriasis. It must be pointed out that manganese is part of enzymes such as superoxide dismutase SOD, which shows a high antioxidant capacity and protection against free radicals, with anti-proliferative effect. In this regard, substances that are part of SOD type enzymes with antioxidant capacity should be seen as technical equivalents of manganese.

The preferred TNF-α anti-proliferative and blocking substance is pentoxifylline, which shall not be applied in the case of acne.

The corticoid is high power, clobetasol propionate for the treatment of vitiligo, alopecia areata, and psoriasis, in a decreasing concentration with decrease of the pathological intensity. The proportions are between 0 and 0.1% by weight of clobetasol propionate.

The corticoid will be of low power, hydrocortisone, and/or medium power, triamcinolone acetonide for treating atopic dermatitis, in a decreasing concentration with decrease in the pathological intensity. It will contain between 0 and 0.5% by weight of triamcinolone acetonide, and between 0 and 2% by weight of hydrocortisone.

For the acute stages of the disease, the formulations include corticoids, although with a tendency to their rapid removal or reduction, and maintenance stages they are deleted from the formulation.

For the treatment of atopic dermatitis, the formula will include up to 5% by weight of doxepin, to block the inflammation, especially in the case of atopic dermatitis. Proportions above 5% could be toxic.

For the treatment of psoriasis varying proportions up to 5% by weight of manganese will be included, because of its important antioxidant and anti-proliferative combined action.

More than one of the topical antioxidants is an activator of PPAR-γ. A non-limitative example is lipoic acid, whose inclusion in a topical formulation is completely new.

Generally, and as it will be appreciated, a very important and innovative characteristic of the formulation of the invention is that it is formulated with a variety of active ingredients that block corresponding receptors for mast cells, to control the multiple inflammation with different simultaneous etiologies, or origin in neurological stress, and with different disease manifestations, possibly simultaneously.

For example, the lipoic acid regulates the peroxisome proliferator-activated receptor gamma (PPAR-γ).

As a regulator of sebaceous secretion proportions of nicotinamide can be included.

As excipient propylene glycol can also be added, which acts as a solvent to increase the solubility of the active ingredients.

### EXAMPLES

### Example 1

### Family of Anti-Inflammation Formulas (Psoriasis, Vitiligo, Alopecia Areata)

A) Common active ingredients (Formula Skeleton).
Corticoids:
   Clobetasol propionate . . . 0.05% (high power)
Indometacin . . . 1-3% (anti-inflammatory)
Pentoxifylline . . . 1-3% (anti-proliferative)
Antioxidants (combinations of):
   Lipoic acid . . . 3-5%
   Quercetin . . . 0.1 to 5%
   Green Tea Catechins . . . 0.5%
   Curcumin . . . 1%
   Ascorbyl Palmitate . . . 5%
   Coenzyme Q10 . . . 0.3%
   Resveratrol . . . 1%
   Pycnogenol® . . . 1%
   L-Carmosine . . . 2%
   Taurine . . . 0.5%
   Isoflavones . . . 5%
   Lycopene . . . 1%
   Papaya . . . Q.s
   Other antioxidants
Excipients: Base Beeler®, Orabase®, water-alcohol solution

B) Additional Active Ingredients
Emollients:
   Glycerin . . . 5-15%
   Aloe Vera . . . 5-15%
   Propilengicol . . . 5-20%
   Lactic Acid . . . 5-12%
   Omega3 . . . 5-10%
Doxepin . . . 1-5%
Ginseng Extract . . . 1-2%
Ketoconazole (antifungal) . . . 0.1-2%
Nicotinamide . . . 2%
L-carnitine . . . 1%
Gentamicin (antibiotic) . . . 0.1%
Manganese (Psoriasis) 0.01 to 5%

### Example 2

### Family of Maintenance Formulas (Psoriasis, Vitiligo, Alopecia Areata)

A) Common active ingredients (Formula Skeleton).
Indometacin . . . 1-3% (anti-inflammatory)
Pentoxifylline . . . 1-3% (anti-proliferative)
Antioxidants (combinations of):
   Lipoic acid . . . 3-5%
   Quercetin . . . 0.1 to 5%
   Green Tea Catechins . . . 0.5%
   Curcumin . . . 1%
   Ascorbyl Palmitate . . . 5%
   Coenzyme Q10 . . . 0.3%
   Resveratrol . . . 1%
   Pycnogenol® . . . 1%
   L-Carmosina . . . 2%
   Taurine . . . 0.5%
   Isoflavones . . . 5%
   Lycopene . . . 1%
   Other antioxidants
Excipients: Base Beeper®, Orabase®, water-alcohol solution

B) Supplementary Active Ingredients.
Emollients:
   Glycerin . . . 5-15%
   Aloe Vera . . . 5-15%
   Propilengicol . . . 5-20%
   Lactic Acid . . . 5-12%
   Omega3 . . . 5-10%
Doxepin . . . 1-5%
Ginseng Extract . . . 1-2%
Ketoconazole (antifungal) . . . 0.1-2%
Nicotinamide . . . 2%
L-carnitine . . . 1%
Gentamicin 0.1% (antibiotic)
Manganese (Psoriasis) . . . 0.01 to 5%

### Example 3

### Family of Anti-Inflammation Formulas (Atopic Dermatitis or Eczema)

A) Common Active Ingredients (Formula Skeleton).
Corticoids:
   Triamcinolone acetonide 0.1% (medium power)
   Hydrocortisone 1% (low power)
Indometacin . . . 1-3% (anti-inflammatory)
Pentoxifylline . . . 1-3% (anti-proliferative)
Antioxidants (combinations of):
   Lipoic acid . . . 3-5%
   Quercetin . . . 0.1 to 5%
   Green Tea Catechins . . . 0.5%
   Curcumin . . . 1%
   Ascorbyl Palmitate . . . 5%
   Coenzyme Q10 . . . 0.3%
   Resveratrol . . . 1%
   Pycnogenol® . . . 1%
   L-Carmosina . . . 2%
   Taurine . . . 0.5%
   Isoflavones . . . 5%
   Lycopene . . . 1%
   Vitamin E . . . 1%
Excipients: Base Beeper®

B) Additional Active Ingredients
Emollients:
   Glycerine . . . 5-15%
   Aloe Vera . . . 5-15%
   Propilengicol . . . 5-20%
   Lactic Acid . . . 5-12%
   Omega3 . . . 5-10%
Doxepin . . . 1-5%
Ginseng Extract . . . 1-2%
Ketoconazole (antifungal) . . . 0.1-2%
Nicotinamide . . . 2%
Gentamicin . . . 0.1% (antibiotic)

### Example 4

### Family of Maintenance Formulas (Atopic Dermatitis, or Eczema)

A) Common Active Ingredients (Formula Skeleton).
Indometacin . . . 1-3% (anti-inflammatory)
Pentoxifylline . . . 1-3% (anti-proliferative)
Antioxidants (combinations of):
   Lipoic acid . . . 3-5%
   Quercetin . . . 0.1 to 5%
   Green Tea Catechins . . . 0.5%
   Curcumin . . . 1%
   Ascorbyl Palmitate . . . 5%
   Coenzyme Q10 . . . 0.3%
   Resveratrol . . . 1%
   Pycnogenol® . . . 1%
   L-Carmosina . . . 2%
   Taurine . . . 0.5%
   Isoflavones . . . 5%
   Lycopene . . . 1%
   Vitamin E . . . 1%
   Other antioxidants
Excipients: Base Beeper®

B) Additional Active Ingredients
Emollients:
   Glycerin . . . 5-15%
   Aloe Vera . . . 5-15%
   Propilengicol . . . 5-20%
   Lactic Acid . . . 5-12%
   Omega3 . . . 5-10%
Doxepin . . . 1-5%
Ginseng Extract . . . 1-2%
Ketoconazole (antifungal) . . . 0.1-2%
Nicotinamide . . . 2%
Gentamicin . . . 0.1% (antibiotic)

### Example 5

### Family of Formulas for Treatment of Acne

A) Common Active Ingredients (Formula Skeleton)
Antibiotic (only one):
   Ciprofloxacin . . . 1%
   Clindamycin . . . 2%
   Sodium Sulfacetamide . . . 10%
   Gentamicin . . . 0.1%
   Erythromycin . . . 2%
Nicotinamide (Vit. PP) . . . 4%
Antioxidants (combinations of):
   Lipoic acid . . . 3-5%
   Quercetin . . . 0.1 to 5%
   Green Tea Catechins . . . 0.5%
   Curcumin . . . 1%
   Ascorbyl Palmitate . . . 5%
   Coenzyme Q10 . . . 0.3%
   Resveratrol . . . 1%
   Pycnogenol® . . . 1%
   L-Carmosina . . . 2%
   Taurine . . . 0.5%
   Isoflavones . . . 5%
   Lycopene . . . 1%
   Vitamin E . . . 1%
   Other antioxidants
Excipient: Hydroalcoholic solution

B) Additional Active Ingredients
Anti-inflammatory: Indometacin
Propilengicol

In each case and for each patient, the doctor will assess—with appropriate regularity—the eventual presence and, where appropriate, the intensity of each of these diseases, and he/she will formulate the composition according to one or the other. For example, if the preeminent disease is psoriasis it will tend to include a greater proportion of manganese, with a higher proportion of ciobetasol propionate with a higher pathological intensity. If the disease is severe atopic dermatitis, it is preferable to formulate with doxepin and acetonide triamcinolone.

As the diagnosis improves, corticoids will be withdrawn gradually, in cases defined in Examples 1 and 3.

Also, the doctor will formulate the combination of topical antioxidants as a function of the intensity of the disease or diseases, which is valued with suitable frequency. Preferred combinations (wt %) of topical antioxidants are:

Low-power combinations for low-intensity inflammations:
Combination 1: 5% ascorbyl palmitate, 5% vitamin E, 3% lycopene.
Combination 2: 1% lycopene, 5% vitamin E, 3% vitamin C.
Medium-power combinations for medium intensity inflammations:
Combination 3: 1% resveratrol
Combination 4: 3-5% lipoic acid
Combination 5: 3% Coenzyme Q10, 5% vitamin E, 5% taurine
Combination 6: q.s. % papaya extract
High-power combinations for intensive inflammations:

### Combination 7: 05% Green Tea Catechins

Combination 8: 1% pycnogenol®, 3% vitamin C, 5% Ascorbyl Palmitate
Combination 9: 1% curcumin.
Combination 10: 2% L-carnosine, 3% vitamin C, 5% vitamin E, 5% Ascorbyl Palmitate
Combination 11: 5% isoflavones, 0.5% green tea catechins, 5% ascorbyl palmitate
Combination 12: 1% quercetin
Other combinations of other antioxidants are also possible.

Therefore, the present invention does not seeks the synergistic effect of the association of two active ingredients to treat a specific disease (e.g topical doxepin and corticoids, as described by Berberian and others), but a multiple synergistic effect on any disease in possible conjunction with other ones arising from new known etiologies, for example allostatic overload by neuroimmunoendocrine stress. Particularly, the topical antioxidants of the combination collaborate synergistically to cause the activation of PPAR-gamma and a consequent improvement of the anti-inflammatory effect. The person skilled in the art will understand that by the reformulation of the formula (% of corticoids and topical antioxidants), the practitioner can gain control of the inflammation, which was not possible in the current state of the art.

With the formulations of the present invention recurrence is reduced, the inflammation, proliferation and infection are controlled, without abuse of toxic substances with side effects, antibiotics, corticoids or doxepin in excess, as its effect is enhanced, surprisingly, on low proportions.

In the case of psoriasis and acne, an antibiotic is always included, because bacteria play an important role in these diseases.

What is claimed is:

1. A dermatological pharmaceutical composition for treating inflammatory diseases of the skin, such as for example dermatitis, atopic dermatitis, vitiligo, alopecia areata, acne, psoriasis and pruritus comprising a base anti-inflammatory, such as indometacin, characterized in that it further comprises:
a combination of topical antioxidants to boost the anti-inflammatory effect, and
one or more optional active ingredients selected alternately from:
at least one corticoid, and
an antibiotic.

2. The dermatological pharmaceutical composition according to claim 1, characterized in that at least one of the topical antioxidants is an activator of peroxisome proliferator-activated receptor gamma (PPAR-γ).

3. The dermatological pharmaceutical composition according to claim 1, characterized in that it comprises at least one antioxidant substance with keratinocyte anti-proliferative effects, and at least one blocking substance of tumor necrosis factor alpha (TNF-α) or other cytokines which trigger the acute phase of the inflammatory reaction, also with anti-proliferative effect.

4. The dermatological pharmaceutical composition according to claim 1, characterized in that said optional active ingredients are corticoids, particularly for the treatment of dermatitis, atopic dermatitis, vitiligo, alopecia areata, psoriasis and pruritus.

5. The dermatological pharmaceutical composition according to claim 1, characterized in that said optional active ingredients comprise an antibiotic for the treatment of acne, as inflammation of the skin.

6. The dermatological pharmaceutical composition according to claim 3, characterized in that said antioxidant substance with anti-proliferative effect is manganese, as part of enzymes such as superoxide dismutase SOD, which have high antioxidant and protection capacity against free radicals with anti-proliferative effect, in a proportion by weight of up to 5%, particularly apt in the case of psoriasis.

7. The dermatological pharmaceutical composition according to claim 3, characterized in that said at least one blocking substance of TNF-α, also with anti-proliferative effect, is pentoxifylline, in a proportion by weight of up to 5%.

8. The dermatological pharmaceutical composition according to claim 4, characterized in that said corticoid is clobetasol propionate, particularly for the treatment of vitiligo, alopecia areata, and psoriasis, in a decreasing concentration with the decrease of the pathological intensity.

9. The dermatological pharmaceutical composition, according to claim 8, characterized in that it contains between 0 and 0.1% by weight of clobetasol propionate.

10. The dermatological pharmaceutical composition, according to claim 4, characterized in that said corticoid is a combination of hydrocortisone and triamcinolone acetonide, particularly for the treatment of dermatitis and atopic dermatitis, in a decreasing concentration with decrease in the pathological intensity.

11. The dermatological pharmaceutical composition according to claim 10, characterized in that it contains between 0 and 0.5% by weight of triamcinolone acetonide.

12. The dermatological pharmaceutical composition, according to claim 10, characterized in that it contains between 0 and 2% by weight of hydrocortisone.

13. The dermatological pharmaceutical composition according to claim 4, characterized in that it contains doxepin, especially for the treatment of atopic dermatitis.

14. The dermatological pharmaceutical composition, according to claim 13, characterized in that it contains between 0 and 5% by weight of doxepin.

15. The dermatological pharmaceutical composition according to claim 1, characterized in that antioxidants of said combination of topical antioxidants are selected from: green tea, lipoic acid, curcumin, ascorbyl palmitate, Coenzyme Q10, resveratrol, pycnogenol®, L-carnosine, taurine, vitamin E, vitamin C, papaya extract, isoflavones, manganese, lycopene, and quercetin.

16. The dermatological pharmaceutical composition, according to claim 15, characterized in that said combination of topical antioxidants is selected from the group consisting of:
5% ascorbyl palmitate, 5% vitamin E, and 3% lycopene,
1% lycopene, 5% vitamin E, and 3% vitamin C,
1% resveratrol,
3-5% lipoic acid,
3% Coenzyme Q10, 5% vitamin E, and 5% taurine
Q.s. % extract of papaya,
0.5% green tea catechins,
1% pycnogenol®; 3% vitamina C; and 5% ascorbyl palmitate,
1% curcumin
2% L-carnosine, 3% vitamin C, 5% vitamin E, and 5% ascorbyl palmitate,
5% isoflavones, 0.5% green tea catechins; 5% ascorbyl palmitate, and
1% quercetin

17. The dermatological pharmaceutical composition, according to claim 1, characterized in that it also includes an emollient selected from: glycerin, aloe vera, propylene glycol, and lactic acid.

18. The dermatological pharmaceutical composition, according to claim 1, characterized in that it also comprises an anti-inflammatory adjuvant, such as omega-3.

19. The dermatological pharmaceutical composition, according to claim 1, characterized in that it further comprises an antifungal, such as ketoconazole.

20. The dermatological pharmaceutical composition, according to claim 1, characterized in that it also comprises L-carnitine.

21. The dermatological pharmaceutical composition according to claim 4, characterized in that it also comprises an antibiotic, such as gentamicin, ciprofloxacin, clindamycin, or the equivalents.

22. The dermatological pharmaceutical composition, according to claim 5, characterized in that said antibiotic is selected from the group consisting of: ciprofloxacin, clindamycin, sulfacetamide sodium, gentamicin and erythromycin.

23. The dermatological pharmaceutical composition, according to claim 1, characterized in that it further comprises nicotinamide.

24. A method of treating a skin inflammation disease comprising administering to a patient in need thereof a dermatological pharmaceutical composition of claim 1.
